(19) United States
(12) Patent Application Publication
(10) Pub. No.: US 2004/0063721 A1
(43) Pub. Date: Apr. 1, 2004

(54) Agonism of the 5HT2A receptor for treatment of thermoregulatory dysfunction

(75) Inventors: Darlene Coleman Deecher, Quakertown, PA (US); Istvan Jozsef Merchenthaler, Chester Springs, PA (US); Liza Leventhal, Lawrenceville, NJ (US); Kimberly Jean Sipe, King of Prussia, PA (US); Lawrence Thomas O'Connor, Des Plaines, IL (US); Terrance Andree, Doylestown, PA (US)

Correspondence Address: WYETH, PATENT LAW GROUP, FIVE GIRALDA FARMS, MADISON, NJ, 07940, US

(73) Assignee: Wyeth, Madison, NJ

(21) Appl. No.: 10/641,226

(22) Filed: Aug. 14, 2003

Related U.S. Application Data
(60) Provisional application No. 60/403,692, filed on Aug. 15, 2002.

(51) Int. Cl. A61K 31/496; A61K 31/445; A61K 31/405; A61K 31/137

(52) U.S. Cl. 514/253.09; 514/317; 514/419; 514/464; 514/651

(57) ABSTRACT

The present invention relates to a method for treating thermoregulatory disorders by administering compounds and compositions of compounds which by modulating 5HT levels activate the 5HT₂ₐ receptor. The invention also relates to therapy using 5HT₁ₐ antagonists and SRIs in combination and pharmaceutical compositions and products containing it. It is emphasized that this abstract is provided to comply with the rules requiring an abstract that will allow a searcher or other reader to quickly ascertain the subject matter of the technical disclosure. It is submitted with the understanding that it will not be used to interpret or limit the scope or meaning of the claims.

### CROSS REFERENCE TO RELATED APPLICATION

This application claims the benefit under 35 U.S.C. §119(e) to U.S. provisional Serial No. 60/403,692 filed Aug. 15, 2002, which is hereby incorporated by reference in its entirety.

### FIELD OF THE INVENTION

The present invention relates a method of treating, preventing, alleviating or inhibiting vasomotor instability and to compositions which achieve such effect. More specifically, the present invention relates to compounds and compositions of compounds and their uses which by modulating serotonin (5HT) levels activate the 5HT₂ₐ receptor. The invention further relates to a method of increasing 5HT signaling through the 5HT₂ₐ receptor using a combination therapy for the treatment of thermoregulatory disorders, using a combination of 5HT₁ₐ antagonist and serotonin reuptake inhibitor (SRI) and pharmaceutical compositions and products containing the same.

### BACKGROUND OF THE INVENTION

It is well recognized that hot flushes are caused by fluctuations of sex steroid levels and they can be disruptive and disabling in both males and females. Hot flush can last up to thirty minutes and vary in their frequency from several times a week to more than a dozen attacks per day. The patient experiences a hot flush as a suddenly occurring feeling of heat that spreads quickly from the face to the chest and back and then over the rest of the body. These attacks are usually accompanied by outbreaks of profuse sweating. They sometimes occur several times an hour, and they often occur at night. Hot flushes and sweating occurring during the night can cause sleep deprivation, resulting in psychological and emotional symptoms such as nervousness, fatigue, irritability, insomnia, depression memory loss, headache, anxiety, nervousness timidity or inability to concentrate. (Murphy et al., 3^{rd} Int'l Symposium on Recent Advances in Urological Cancer Diagnosis and Treatment-Proceedings. Paris, France: SCI: 3-7 (1992)).

Hot flushes may be even more severe in women who have survived breast cancer for several reasons: 1) many survivors of breast cancer are given tamoxifen, the most prevalent side effect of which is hot flush, 2) many women treated for breast cancer undergo premature menopause from chemotherapy, 3) women with a history of breast cancer have generally been denied estrogen therapy because of concerns about potential recurrence of breast cancer (Waldinger et al, Maturitas, 2000, 36(3): p. 165-168.).

Men also experience hot flushes following steroid hormone (androgen) withdrawal. This is true in cases of age-associated androgen decline (Katovich et al., Proceedings of the Society for Experimental Biology & Medicine, 1990. 193(2): p. 129-35) as well as in extreme cases of hormone deprivation associated with treatments for prostate cancer (Berendsen et al, European Journal of Pharmacology, 2001. 419(1): p. 47-54). As many as one-third of these patients will experience persistent and frequent symptoms severe enough to cause significant discomfort and inconvenience.

Menopausal hot flushes are most commonly treated with hormone replacement therapy (orally, transdermally, or via an implant), however some patients cannot tolerate estrogen or androgen treatment (Berendsen, Maturitas, 2000. 36(3): p.155-164, Finket al., Nature., 1996. 383(6598): p. 306). In addition, hormone replacement therapy is usually not recommended for women or men with or at risk for hormonally sensitive cancers (e.g. breast or prostate cancer). Thus, non-steroidal therapies (e.g. fluoxetine, paroxetine and clonidine) are being evaluated clinically. WO9944601 discloses a method for decreasing hot flushes in a human female by administering fluoxetine. Other options have been studied for the treatment of hot flushes, including steroids, alpha-adrenergic agonists, and beta-blockers, with varying degree of success (Waldinger et al., Maturitas, 2000. 36(3): p. 165-168).

Berendsen et al, (European Journal of Pharmacology, 2001, 419(1): p. 47-54), and Fink, G., et al., Clinical & Experimental Pharmacology & Physiology, 1998. 25(10): p. 764-75) have reported that 5-HT₂ₐ receptor antagonists are indicated for treatment of hot flushes.

Hot flushes are a source of great physical and mental stress. While the balance of sex hormones and neurotransmitters is critical for maintaining normal thermoregulation, the mechanisms involved in thermoregulatory dysfunction are, per se largely unknown. Thermoregulatory disorders often warrant medical treatment, yet a satisfactory treatment having few side effects has not been forthcoming. Accordingly, given the multifaceted nature of thermoregulation, multiple therapies and approaches can be used to target vasomotor instability.

The present invention, focuses on novel mechanisms that promote 5HT₂ₐ receptor signaling to alleviate vasomotor instability.

### SUMMARY OF THE INVENTION

The present invention provides a method of treating a subject suffering from or susceptible to thermoregulatory disorders which method comprises administering to a subject a therapeutically effective amount of one or more compounds which agonize the 5HT₂ₐ receptor.

Also provided are methods of agonizing the 5HT₂ₐ receptor by endogenously produced compounds such as serotonin. The present invention includes a method where endogenously produced serotonin is modulated by a 5HT₁ₐ antagonist and an SRI. In some aspects of the invention 5HT₁ₐ antagonist and an SRI may be provided in by a single compound having dual activity. Also provided is a method using a combination therapy comprising administering to a subject an effective amount of a first component which is a 5HT₁ₐ antagonist, its derivatives and or pharmaceutically acceptable salts thereof in combination with an effective amount of a second component which is a serotonin reuptake inhibitor, its derivatives and or pharmaceutically acceptable salts thereof. In yet another embodiment is provided a method of agonizing 5HT₂ₐ receptor by administering any of the compounds such as fluoxetine, paroxetine, sertraline, and fluvoxamine, amoxapine, doxepin, bupropion, citalopram, and amitriptyline with a 5HT₁ₐ antagonist.

In one embodiment, 5HT₂ₐ agonist is selected from the group consisting of 1(2,5-dimethoxy-4-iodophenyl)-2-aminopropane hydrochloride (DOI) and (±)-2,5-dimoethoxy-4-bromoamphetamine hydrobromide (DOB). In another embodiment, the SRI is selected from the group consisting of fluoxetine, paroxetine, sertraline, fluvoxamine, duloxetine, amoxapine, doxepin, bupropion, citaloprom and amitriptyline. In yet another embodiment, 5HT₁ₐ antagonist is selected from the group consisting of N-[2-[4-(2-Methoxyphenyl)-1-piperazinyl]ethyl]-N-2-pyridinylcyclohexanecarboxamide (WAY-100635), (R)-N-(2-Methyl-(4-indolyl-1-piperazinyl)ethyl)-N-(2-pyridinyl)cyclohexanecarboxamide and NAD-299 (Astra Zeneca). A preferred 5HT₁ₐ antagonist is WAY-100635.

A method is also provided wherein the 5HT₂ₐ receptor is agonized by exogenously administered compound.

### BRIEF DESCRIPTION OF THE DRAWINGS

The invention can be more fully understood from the following detailed description and the accompanying drawings, which form a part of this application.

FIG. 1A and 1B depict the effect of 5HT₂ₐ receptors in the thermoregulation (referred to in Example 1).

FIG. 2A and 2B demonstrate that 5HT_{2c} receptors are not involved in thermoregulation in a morphine-dependent rat model of vasomotor instability (* indicates p<0.05 compared to vehicle control) (referred to in Example 2).

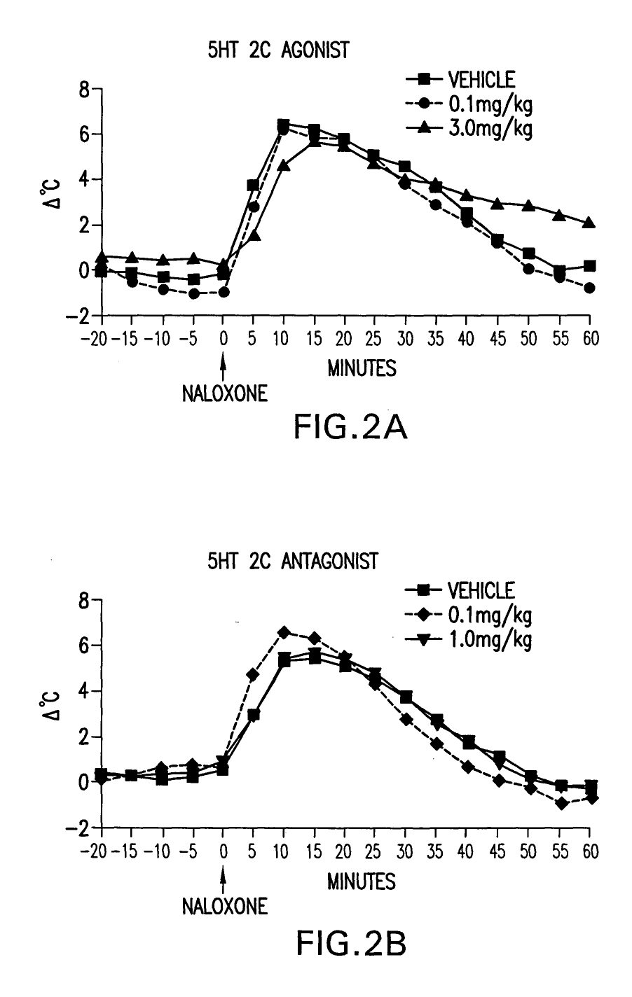

FIG. 3A and 3B show the effect of redirecting 5HT signaling to 5HT₂ₐ receptor using a combination of SRI and 5HT₁ₐ in alleviation of hot flush (* indicates p<0.05 compared to vehicle control) (referred to in Example 3).

FIG. 4A and 4B show the effect of 5HT₁ₐ receptor antagonism in combination with SRI in alleviation of hot flush (referred to in Example 4). FIG. 4A: * indicates p<0.05 compared to vehicle control; Ω indicates p<0.05 compared to fluoxetine 10 mg/kg. FIG. 4B: * indicates p<0.05 compared to vehicle control; Ψ indicates p<0.05 compared to WAY-100635 0.1 mg/kg; Φ indicates p<0.05 compared to WAY-100635 1.0 mg/kg.

FIG. 5A show the ability of a 5HT₂ₐ receptor antagonist (MDL-100907) to block the combination of a SRI, such as fluoxetine, and 5HT₁ₐ receptor antagonist (WAY-100635). FIG. 5B depicts a dose (10 mg/kg) of fluoxetine (an SRI compound) that is ineffective at alleviating hot flush but is potentiated by 5HT₂ₐ receptor antagonist. FIG. 5C demonstrates that an effective dose (30 mg/kg) of fluoxetine (an SRI compound) is similarly potentiated by the 5HT₂ₐ receptor antagonist (referred to in Example 5). * indicates p<0.05 compared to vehicle control; Φ indicates p<0.05 compared to 0.01 mg/kg MDL-100907; Ψ indicates p<0.05 compared to 0.1 mg/kg MDL-100907.

### DETAILED DESCRIPTION OF THE INVENTION

The present invention comprises methods of treating, preventing, alleviating or inhibiting vasomotor instability in a subject, preferably in human subject, the method comprising administering a therapeutically effective amount of one or more compounds which agonize the 5HT₂ₐ receptor. The 5HT₂ₐ receptor may be agonized by endogenously or exogenously produced compounds. The present invention further comprises methods wherein endogenously produced compound is serotonin. The endogenously produced serotonin may be modulated by combination of 5HT₁ₐ antagonist and SRI and pharmaceutical compositions and products containing the same.

The present invention is useful for treating menopause-induced hot flush, chemical or surgical induced steroid deprivation (cancer survivors), androgen ablation therapies and anti-estrogen therapies and the like. The present invention is particularly useful for patients who are unable to take steroid-based therapies.

The following definitions are provided for the full understanding of terms and abbreviations used in this specification.

The abbreviations in the specification correspond to units of measure, techniques, properties or compounds as follows: “min” means minutes, “h” means hour(s), “μL” means microliter(s), “mL” means milliliter(s), “μM” means micromolar, “mM” means millimolar, “M” means molar, “mmole” means millimole(s), “SEM” means standard error of the mean and “IU” means International Units. “Δ° C.” means change in tail skin temperature normalized for 15 min baseline TST prior to naloxone-induced flush. “ΔTST” means change in TST from baseline, and “ED₅₀ value” means dose which effects 50% of the population for endpoint readout.

“Tail skin temperature” is abbreviated TST.

“Serotonin reuptake inhibitor” is abbreviated SRI. Selective serotonin reuptake inhibitors are a class of SRIs and are abbreviated SSRI. Examples of SRIs include, but are not limited to, of fluoxetine, paroxetine, sertraline, duloxetine, fluvoxamine, amoxapine, doxepin, bupropion, citalopram and amitriptyline.

“Serotonin” is abbreviated 5HT.

“Subcutaneous” is abbreviated sc.

In the context of this disclosure, a number of terms shall be utilized. The term “treatment” as used herein includes preventative (e.g., prophylactic), curative or palliative treatment and “treating” as used herein also includes preventative, curative and palliative treatment.

As used herein, the term “subject” refers to an animal including the human species that is treatable with the compositions, methods of the present invention. The term “subject” or “subjects” is intended to refer to both the male and female gender unless one gender is specifically indicated.

The term “patient” comprises any animal, which may benefit from treatment or prevention of vasomotor disturbances. The term patient comprises female animals including humans and, among humans, not only women of advanced age who have passed through menopause but also women who have passed through premature menopause, undergone hysterectomy or for some other reason have suppressed estrogen production, such as those who have undergone long-term administration of corticosteroids, suffer from Cushions' syndrome or have gonadal dysgenesis. The term “patient” is not intended to be limited to a woman.

The term “premature menopause” refers to ovarian failure of unknown cause that may occur before age 40. It may be associated with smoking, living at high altitude, or poor nutritional status. Artificial menopause may result from oophorectomy, chemotherapy, radiation of the pelvis, or any process that impairs ovarian blood supply.

Ovariectomy means removal of an ovary or ovaries that result in steroid deprivation (Merchenthaler et al, Maturitas, 1998, Nov 16; 30(3): 307-316).

The term “premenopausal” means before the menopause, the term perimenopausal means during the menopause and the term postmenopausal means after the menopause.

The term “hot flush” is an art recognized term that refers to an episodic disturbance in body temperature typically consisting of a sudden elevation in skin temperature, usually accompanied with sweating and perspiration in a subject.

The term “hot flush” may be used interchangeably with the terms vasomotor instability, vasomotor dysfunction, thermoregulatory disorders, night sweats, hot flash, vasomotor disturbances, or vasomotor disorders.

A “therapeutically effective amount” refers to an amount effective, at dosages and for periods of time necessary, to achieve the desired result. It will be appreciated that the therapeutically effective amount of components of the present invention will vary from patient to patient not only with the particular compound, component or composition selected, the route of administration, and the ability of the components (alone or in combination with one or more combination drugs) to elicit a desired response in the individual, but also with factors such as the disease state or severity of the condition to be alleviated, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the optimum therapeutic response. A therapeutically effective amount is also one in which any toxic or detrimental effects of the components are outweighed by the therapeutically beneficial effects.

Preferably, the compounds of the present invention are administered at a dosage and for a time such that the number of hot flushes is reduced as compared to the number of hot flushes prior to the start of treatment. Such treatment can also be beneficial to reduce the overall severity or intensity distribution of any hot flushes still experienced, as compared to the severity of hot flushes prior to the start of the treatment. The subject, preferably human, may be female, and more preferably perimenopausal, monopausal or post-menopausal. Male patients which are andropausal may also be treated in accordance with the present invention.

A pharmaceutical for use in accordance with the present invention comprises, a 5HT₂ₐ agonist, a combination of 5HT₁ₐ antagonist and serotonin reuptake inhibitor in a single compound having dual activity, or a combination of compounds, and pharmaceutically acceptable salt thereof, together with a pharmaceutically acceptable carrier. The composition may comprise one or more 5HT₂ₐ agonist(s), or one or more each of serotonin reuptake inhibitor(s) and 5HT₁ₐ antagonist(s) as active ingredient(s), or one or more of a compound having both serotonin reuptake inhibitor(s) activity and 5HT₁ₐ antagonist(s) activity, together with one or more pharmaceutically acceptable carrier(s).

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule or peptide.

The term “inhibit” refers to the act of diminishing, suppressing, alleviating, preventing, reducing or eliminating, whether partial or whole, a function or an activity. The term “inhibit” can be applied to both in vitro as well as in vivo systems. As used herein, the term “inhibitor” refers to any agent that inhibits.

Within the present invention, the 5HT₁ₐ antagonist and serotonin reuptake inhibitor may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sufloric acids, and most preferably is the hydrochloride salt.

Further, the compounds of the present invention may exist in unsolvated as well as in solvated forms with pharmaceutically acceptable solvents such as water, ethanol, and the like. In general, the solvated forms are considered equivalent to the unsolvated forms for the purpose of the present invention.

Some of the compounds of the present invention may contain chiral centers and such compounds may exist in the form of isomers (i.e. enantiomers). The present invention includes all such isomers and any mixtures thereof including racemic mixtures.

The route of administration may be any route, which effectively transports the 5HT₂ₐ agonist or, 5HT₁ₐ antagonist and serotonin reuptake inhibitor to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of 5HT₁ₐ antagonist(s) and serotonin reuptake inhibitor(s) may be concurrent or simultaneous.

The term “combination therapy” refers to the administration of two or more therapeutic agents or compounds to treat a therapeutic condition or disorder described in the present disclosure, for example hot flush, sweating, thermoregulatory -related condition or disorder, or other. Such administration includes co-administration of these therapeutic agents or compounds in a simultaneous manner, such as in a single compound having both 5HT₁ₐ antagonist and serotonin reuptake inhibitor activity or in multiple, separate compounds for each 5HT₁ₐ antagonist, and serotonin reuptake inhibitor activities. In addition, such administration also includes use of each type of therapeutic agent in a concurrent manner. In either case, the treatment regimen will provide beneficial effects of the drug combination in treating the conditions or disorders described herein.

The term “central nervous system” or “CNS” includes the brain and the spinal cord. The term “peripheral nervous system” or “PNS” includes all parts of the nervous system that are not part of the CNS, such as cranial and spinal nerves and the autonomic nervous system.

The terms “component”, “drug”, “pharmacologically active agent”, “agent”, and “medicament” are used interchangeably herein to refer to a compound or compounds, e.g., antibody, small molecule, nucleic acid molecule, peptide, oligopeptide, polypeptide, or protein, or compositions containing such, which when administered to an organism (human or animal) induces a desired pharmacologic and/or physiologic effect by local and/or systemic action. The agents herein may act directly at the 5HT₂ₐ receptor or provide combined serotonin reuptake inhibiting effect and 5HT₁ₐ antagonistic effect to modulate signaling of the 5HT₂ₐ receptor.

The activity of the 5HT₂ₐ receptor may be agonized endogenously by an endogenous agonist, such as 5-HT, or exogenously by administration of a 5HT₂ₐ agonistic agent, such as a drug or other synthetic ligand. “5HT₂ₐ receptor activity” includes activity induced by: (1) endogenous agonist; (2) exogenous agonist; and (3) a combination of endogenous and exogenous agonists. Furthermore, the activation of the receptor may be due to the constitutive activation associated with a native, mutated or modified receptor. The receptor may be purified or present in an in vitro or in vivo system.

The agents of the present invention may be administered on an as needed basis or on a continuous regimen. The length of treatment needed to observe changes and the interval following treatment for responses to occur may vary depending on the desired effect. For combination therapy with more than one agent, the agents may be administered concurrently or they can be administered at separately staggered times.

In one embodiment, 5HT₂ₐ antagonists induced flushing in a model of rodent thermoregulation, whereas a 5HT₂ₐ agonist unexpectedly abated this induced flush. It has been found however that some 5HT₂ₐ agonists have undesireable hallucinagenic side effects. Thus, in other embodiments of the invention, agonists having relatively lower affinity than compounds such as DOI or DOB, partial agonists or endogenous agonists of the 5HT₂ₐ receptor eliminate the undesirable side effects of a 5HT₂ₐ agonists while retaining the therapeutic benefit of alleviating vasomotor symptoms. For instance, 5HT levels may be modulated thereby activating the 5HT₂ₐ receptor through its endogenous ligand.

In another embodiment, 5-HT₂ₐ receptor agonism using DOI (5-HT_{2a/2c} agonist) had the desired effect of preventing a naloxone-induced increase in tail-skin temperature. DOI and DOB are non-limiting examples of 5HT₂ₐ agonists. This effect is similar to those observed for other clinical therapies for hot flush that have been evaluated in this model (Merchenthaler et al, Maturitas., 1998. 30(3): p. 307-16). Furthermore, 5-HT_{2c} agonists and antagonists had no effect in this model, indicating the 5-HT₂ₐ receptor is being modulated.

In yet another embodiment, increased activation of the 5-HT₂ₐ receptor by its endogenous ligand, serotonin (5-HT), provides a means of alleviating hot flush (normalizing thermoregulation) without undesirable hallucinogenic side-effects. A combination therapy including 5-HT₁ₐ receptor antagonist and SRI was able to increase 5HT signaling through the 5HT₂ₐ receptor sufficient to alleviate hot flush.

In yet another embodiment, 5HT₂ₐ agonist is selected from the group consisting of 1-(2,5-dimethoxy-4-iodophenyl)-2-aminopropane hydrochloride (DOI) and (±)-2,5-dimoethoxy-4-bromoamphetamine hydrobromide (DOB). In another embodiment, the SRI is selected from the group consisting of fluoxetine, paroxetine, sertraline, fluvoxamine, duloxetine, amoxapine, doxepin, bupropion, citaloprom and amitriptyline. In yet another embodiment, 5HT₁ₐ antagonist is selected from the group consisting of N-[2-[4-(2-Methoxyphenyl)-1-piperazinyl]ethyl]-N-2-pyridinylcyclohexanecarboxamide (WAY-100635), (R)-N-(2-Methyl-(4-indolyl-1-piperazinyl)ethyl)-N-(2-pyridinyl)cyclohexanecarboxamide and NAD-299 (Astra Zeneca).

In a preferred embodiment, compounds act through peripheral mode of action on 5HT₂ₐ receptors.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

GENERAL METHODS

Reagents

MDL-100907 (5HT₂ₐ antagonist) was synthesized as described in WO91/18602. The following reagents were purchased commercially: fluoxetine (SRI, Sigma),1-(2,5-dimethoxy-4-iodophenyl)-2-aminopropane (5HT₂ₐ agonist, DOI, Sigma), WAY-100635 (5HT₁ₐ antagonist, described in U.S. Pat. No. 6,127,357, which is hereby incorporated by reference), morphine alkaloid pellets (Murty Pharmaceuticals, Lexington, Ky.), ketamine (Phoenix Pharmaceuticals, Belmont, Calif.), and naloxone (Research Biochemicals International, St. Louis, Mo.).

Dosing

All compounds were dissolved in sterile water except MDL-100907 which was dissolved in Tween 80, injected subcutaneously (sc) and administered at the following dosages: fluoxetine (10 mg/kg), WAY-100635 (0.01, 0.1, and 1.0 mg/kg), DOI (1 mg/kg), MDL-100907 (0.3 and 1 mg/kg) and naloxone (1.0 mg/kg). Ketamine (Ketaject, PhoenixPharmaceuticals, Belmont, Calif.) was administered intramuscularly at 40 mg/kg at a dose that was determined to be mildly sedative but did not cause a change in tail skin temperature (TST).

Animals

Ovariectomized Sprague-Dawley rats (180-220 g) were obtained from a commercial vendor (Taconic, Germantown, N.Y.) and individually housed under 12 h light/dark cycle in a room maintained at 25° C. Animals were provided with standard rat chow and water ad libitum.

Morphine-dependent Experimental Model

Ovariectomized rats were injected once daily for 8-9 days with vehicle to minimize stress responses and then administered compound(s) on test day. On day 4 of dosing, morphine dependence was induced by subcutaneous (sc) implantation of two slow-release morphine pellets (75 mg/pellet) in the dorsal scapular region. This model is based upon an established morphine-dependent naloxone-induced flush paradigm that is reversible by estrogen treatment (Katovich et al., Proceedings of the Society for Experimental Biology & Medicine, 1990.193(2): p.129-35). Four to six days after implantation, morphine withdrawal was induced with an opioid antagonist (naloxone) that causes a transient increase in TST. In a typical experiment, rats were administered their final dose of test compound 1 h prior to naloxone injection. Rats were mildly sedated with ketamine and a thermistor connected to a MacLab data acquisition system was taped to the base of the tail. Tail skin temperature was then monitored continuously for 35 minutes to establish a baseline temperature. Naloxone was subsequently administered and TST was measured for an additional 60 min (total recording time 95 min).

OVX-induced Thermoregulatory Dysfunction Model

To determine the effect test compounds had on thermoregulation, TST was monitored in ovariectomized rats by telemetry. This model has been modified from a previously reported protocol based on estrogen regulation of diurnal TST patterns (Berendsen et al.European Journal of Pharmacology, 2001. 419(1): p.47-54). Following acclimation, a temperature and physical activity transmitter (PhysioTemp TA10TA-F40, Data Sciences International) was implanted sc in the dorsal scapular region and the tip of temperature probe was tunneled sc into the tail and secured 2 cm beyond the base. After a 7 day recovery period, baseline TST recordings were monitored for up to 3 days to establish a baseline. Rats were then administered a single dose of either test compound or vehicle and TST was monitored continuously for up to 12 h. Since TST varies between the active (dark) and inactive (light) phase over a 24 h period, effects of test compounds were tested during each of these phases by dosing during the light cycle or just prior to dark cycle.

Statistical Analysis

To analyze changes in TST induced by naloxone in morphine-dependent rats, all data was analyzed using a two factor repeated measure. The factors were “treatment” and “time” (repeated). The model was fit to test whether there were significant differences in the responses between treatment groups. The data was analyzed at 5 minute intervals from 20 minutes (−20) prior to the naloxone administration (referred to as time 0) to 60 minutes after the treatment. The first three readings were averaged and used as baseline TST scores. All data was analyzed as ΔTST (TST for each time point—baseline). Multiple comparisons (least square deviation (LSD) p-values) among the treatment groups at each time point were used for the analysis, however, the changes in TST is greatest at 15 minutes post naloxone administration and this time point provides the best indicator of flush abatement. When appropriate, estimation of the ED₅₀ value was calculated. The ED₅₀ value was determined using a log scale and the line was fit between the maximal (15 min post naloxone ΔTST) and minimal response (average baseline temperature prior to naloxone). The ED₅₀ value is reported as the dose of test compound that abates 50% of the naloxone- induced flush.

To analyze changes in TST induced by test compounds in the thermoregulatory dysfunction model, a repeated measures analysis was performed. The model used for analysis was TST=((GRP (group)+HR (hours))+((GRP*HR)+BASELINE)), using an unstructured error covariance matrix. Thus, the reported least square means are the expected mean values as if both groups had the same baseline value. Post-hoc tests of hourly GRP*HR intervals are essentially t-tests of a difference between groups for each hour. To be conservative, a result was not considered significant unless the p-value was <0.025. All analyses were performed using SAS PROC MIXED (SAS, Carey, N.C.). For each compound, a baseline temperature for each rat was estimated by averaging temperature readings over a 12 h period on the day prior to dosing.

### EXAMPLE 1

### 5HT₂ₐ Receptor Effect on Thermoregulation

Rats were injected subcutaneously (sc) with vehicle (sterile H20), DOI (5HT_{2a/2c} agonist, Sigma) (dissolved in sterile H20 and administered at 1 mg/kg), 5HT₂ₐ antagonist MDL-100907 was synthesized as described in U.S. Pat Nos. 5,134,149, 5,561,144, 5,700,812, 5,700,813, 5,721,249, 5,874,445, and 6,004,980, which are incorporated herein by reference, dissolved in sterile H20 and administered at 0.3, 1 mg/kg. All drugs were administered 20 min prior to naloxone.

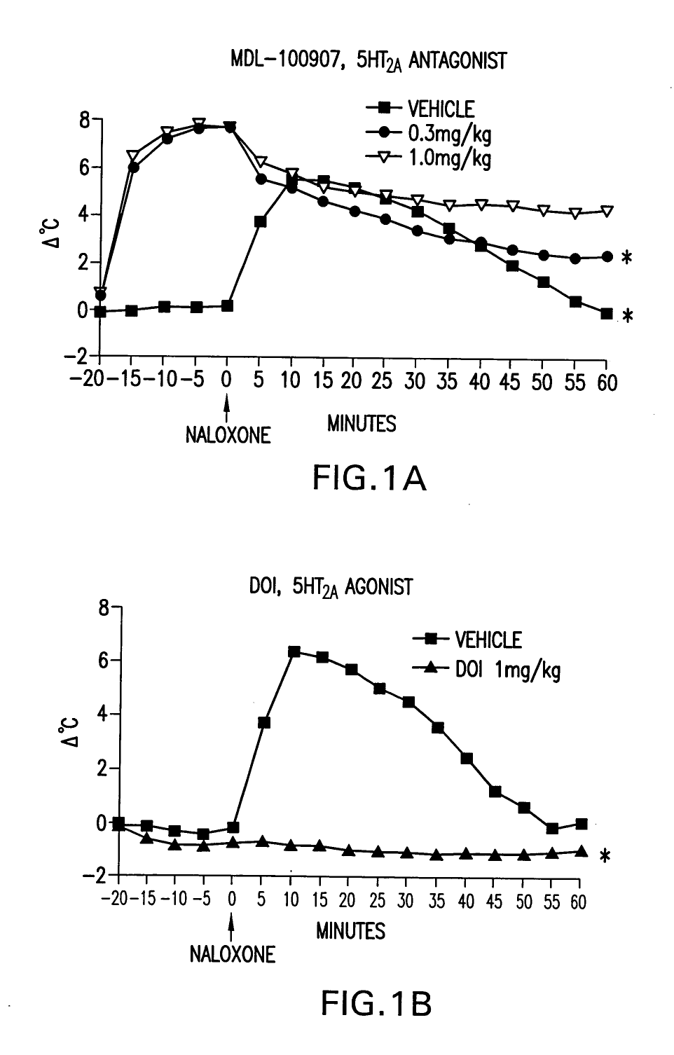

MDL-100907 (5HT₂ₐ antagonist) significantly induced an increase in tail skin temperature prior to administration of naloxone (FIG. 1A). In contrast, DOI (5HT_{2a/2c} agonist) significantly abated the naloxone-induced flush without affecting basal tail skin temperature (FIG. 1B). These data demonstrated that the ability of DOI to alleviate vasomotor instability is selectively mediated through the 5HT₂ₐ receptor.

### EXAMPLE 2

### 5HT_{2c} Receptors and Thermoregulation in Two Rat Models of Vasomotor Instability

Method as described in the general method section with the following exceptions:

Rats were injected subcutaneously with vehicle (sterile H₂O), (7bR, 10aR)-1,2,3,4,8,9,10,10a-octahydro-7bH-cyclopenta[b][1,4]diazepino[6,7,1-hi]indole (5HT_{2c} agonist) was synthesized as described in WO 02/42304, dissolved in sterile H20 and administered at 0.1, 3.0 mg/kg, 5HT_{2c} antagonist (6-Chloro-5-methyl-1-[[2-[(2-methyl3-pyridyl)oxy]-5-pyridyl]carbamoyl]indoline) (Bromidge et al., J. Med. Chem., 1997, 40, 3494-3496) was dissolved in sterile H20 and administered at 0.1, 1.0 mg/kg). All drugs were administered 20 min prior to naloxone.

Changes in TST (Δ° C., Mean) over time in the morphine-dependent rat model depicts (7bR, 10aR)-1,2,3,4,8,9,10,10a-octahydro-7bH-cyclopenta[b][1,4]diazepino [6,7,1-hi]indole (5HT_{2c} agonist) (Panel A) or 6-Chloro-5-methyl-1-[[2-[(2-methyl-3-pyridyl)oxy]-5-pyridyl]carbamoyl]insoine (5HT_{2c} antagonist) (Panel B) did not significantly abate naloxone-induced flush. The lack of activity of 5HT_{2c} receptorspecific compounds indicates the effect of DOI (5HT₂ₐ agonist) is due to its action at the 5HT₂ₐ receptor.

### EXAMPLE 3

### Redirecting 5HT Signaling to 5HT₂ₐ Using a Combination of SRI and 5HT₁ₐ Receptor Antagonist

Rats were injected subcutaneously with vehicle (sterile H20), fluoxetine (Sigma, St Louis, Mo.) was dissolved in sterile H20 at 10 mg/kg), 5HT₁ₐ antagonist, WAY-100635 (Sigma, St Louis, Mo.) was dissolved in sterile H20 and administered at 1 mg/kg or with a combination of fluoxetine and WAY-100635. Fluoxetine was administered 1 h prior to naloxone injection and WAY-100635 was administered 20 min prior to naloxone.

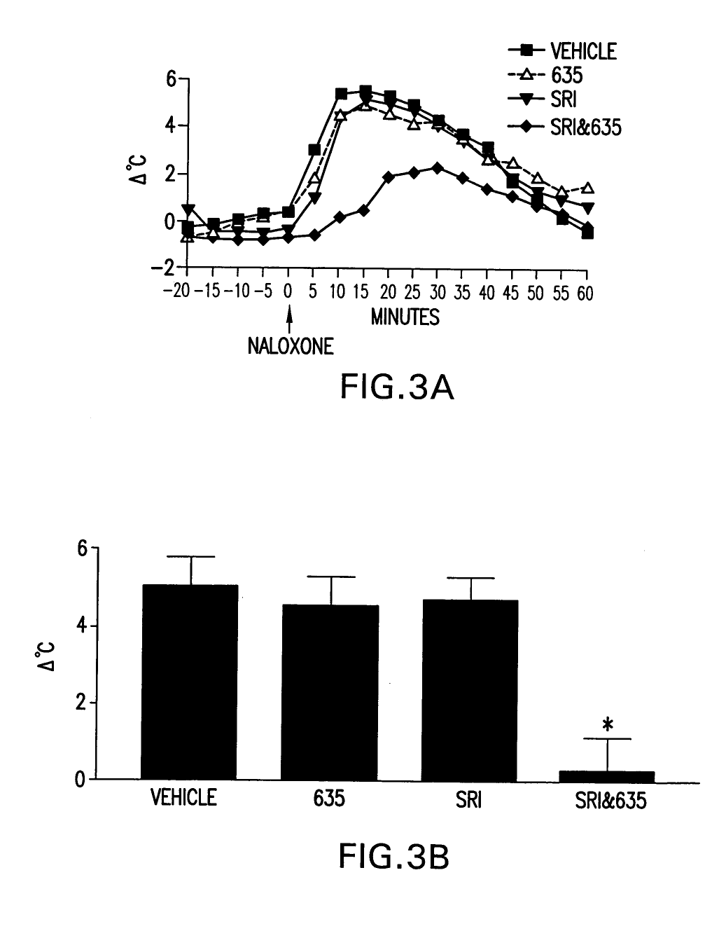

Changes in TST (Δ° C., Mean) over time in the morphine-dependent rat model depict fluoxetine or WAY-100635 administered alone did not significantly abate naloxone-induced flush (FIG. 3A). However, a combination of fluoxetine and WAY-100635 significantly reduced the naloxone-induced flush. At maximal flush (15 min post-naloxone; Δ° C., Mean±SEM) only the combination treatment abated the naloxone-induced flush (FIG. 3B). These data show the synergism between an SRI and 5HT₁ₐ receptor antagonist in alleviating vasomotor instability. In addition an increase in stereotypical “wet-dog shakes” associated with 5HT₂ₐ receptor activation was noted. These data imply that the combination redirects 5HT signaling through the 5HT₂ₐ receptor which is not a generalized phenomenon of 5HT action.

### EXAMPLE 4

Effect of 5HT₁ₐ Receptor Antagonism in Combination with SRI

Rats were injected subcutaneously with vehicle (sterile H20) or fluoxetine (Sigma, dissolved in sterile H20 at 0.1, 1.0 or 10 mg/kg) and the 5HT₁ₐ antagonist WAY-100635 (dissolved in sterile H20 and administered at 1.0 mg/kg) or fluoxetine (10 mg/kg) and WAY-100635 (0.01, 0.1 or 1.0 mg/kg). Fluoxetine was administered 1 h prior to naloxone injection and WAY-100635 was administered 20 min prior to naloxone.

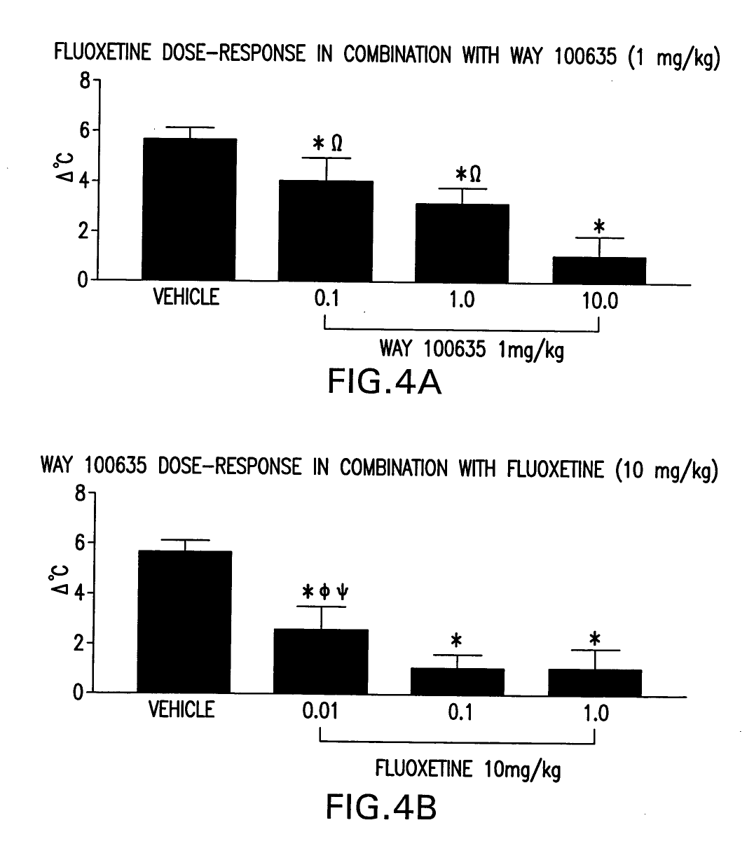

At maximal flush (15 min post-naloxone; Δ° C., Mean±SEM) fluoxetine dosedependently abates the naloxone-induced flush when combined with WAY-100635 (1.0 mg/kg) (FIG. 4A), ED₅₀ value=0.20±0.11. At maximal flush (15 min post-naloxone; Δ° C., Mean±SEM) WAY-100635 dose-dependently abates the naloxone-induced flush when combined with fluoxetine (10 mg/kg) (FIG. 4B), ED₅₀ value=0.01±0.009. These data indicate that 5HT_{1A} receptor antagonist is an important contributor in potentiating the acute action of the SRI since it is capable of promoting fluoxetine action at lower doses. The increased efficacy of fluoxetine (10 mg/kg) in the presence of WAY-100635 is likely the result of more efficient redirecting of 5HT signaling through the 5HT₂ₐ receptor.

### EXAMPLE 5

### 5HT₂ₐ Antagonism

Rats were injected subcutaneously with vehicle (sterile H₂0) or fluoxetine (Sigma, dissolved in sterile H₂0 at 10 mg/kg) and the 5HT₁ₐ antagonist WAY-100635 dissolved in sterile H₂0 and administered at 0.01 mg/kg) or fluoxetine (10 or 30 mg/kg) and MDL-100907 (5HT₂ₐ antagonist, at 0.01 or 0.1 mg/kg). MDL-100907 was administered 55 minutes prior to the naloxone injection followed 15 min later by the administration of either fluoxetine or the combination (Fluoxetine 10 mg/kg±WAY-100635 0.01 mg/kg).

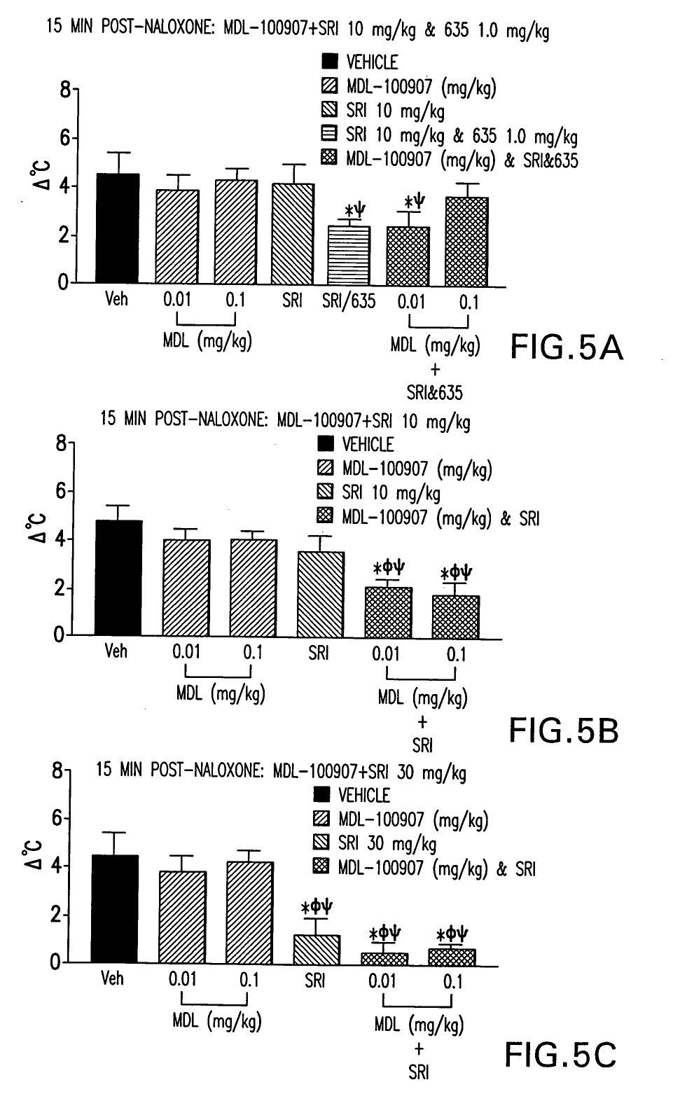

At maximal flush (15 min post-naloxone; Δ° C., Mean±SEM) MDL-100907 dose-dependently reversed the combination drug abatement of the naloxone-induced flush (FIG. 5A). In contrast, at maximal flush (15 min post-naloxone; Δ° C., Mean±SEM) MDL-100907 potentiated the ineffective dose of fluoxetine (10 mg/kg) (FIG. 5B). Additionally, administering MDL-100907 prior to a dose of fluoxetine (30 mg/kg) that abates a naloxone-induced hot flush also potentiated the abatement effect.

These data demonstrate that the 5HT_{2A} receptor antagonism differentially effects the combination fluoxetine (10 mg/kg)/WAY-100635 and fluoxetine 10 or 30 mg/kg. Thus, 5HT_{1A} receptor antagonism in combination with fluoxetine differentiates itself from administration of both low and high doses of fluoxetine suggesting a different mode of action.

What is claimed is:

1. A method of treating a subject suffering from or susceptible to thermoregulatory disorder which method comprises administering to said subject a therapeutically effective amount of one or more compounds which agonize the 5HT₂ₐ receptor.

2. The method of claim 1 wherein the 5HT₂ₐ receptor is agonized by endogenously produced ligand.

3. The method of claim 2 wherein the endogenously produced ligand is serotonin.

4. The method of claim 3 wherein the endogenously produced serotonin is modulated by a 5HT₁ₐ antagonist and an SRI.

5. The method of claim 4 wherein the compound has dual activity comprising 5HT₁ₐ antagonist and SRI activity.

6. The method of claim 4 wherein the 5HT₁ₐ antagonist and SRI are administered as a combination therapy comprising administering to said patient an effective amount of a first component which is a 5HT₁ₐ antagonist, its derivatives and or pharmaceutically acceptable salts thereof in combination with an effective amount of a second component which is a serotonin reuptake inhibitor, its derivatives and or pharmaceutically acceptable salts thereof.

7. The method of claim 6 where the first component is WAY-100635, (R)-N-(2-Methyl-(4-indolyl-1-piperazinyl)ethyl)-N-(2-pyridinyl)cyclohexancaroxamide and NAD-299.

8. The method of claim 6 where the second component is selected from the group consisting of fluoxetine, paroxetine, sertraline, fluvoxamine, duloxetine, amoxapine, doxepin, bupropion, citalopram, and amitriptyline.

9. The method of claim 6 wherein,
a. the first component is WAY-100635, (R)-N-(2-Methyl-(4-indolyl-1-piperazinyl)ethyl)-N-(2-pyridinyl)cyclohexanecarboxamide and NAD-299.
b. the second component is selected from the group consisting of fluoxetine, paroxetine, sertraline, fluvoxamine, duloxetine, amoxapine, doxepin, bupropion, citalopram, and amitriptyline.

10. The method of claim 1 wherein said 5HT₂ₐ agonist is selected from the group consisting of 1-(2,5-dimethoxy-4-iodophenyl)-2-aminopropane hydrochloride (DOI), and (±)-2,5-dimethoxy-4-bromoamphetamine hydrobromide (DOB).

11. The method of claim 6 wherein the administration of 5HT₁ₐ antagonist and serotonin reuptake inhibitor is simultaneous.

12. The method of claim 1 where said subject is human.

13. The method of claim 12 where the human is female patient.

14. The method of claim 13 wherein the female patient is perimenopausal

15. The method of claim 13 where the female patient is menopausal.

16. The method of claim 13 where the female patient is post-menopausal.

17. The method of claim 12 where the human is male.

18. The method of claim 17 where the male patient is naturally, chemically or surgically andropausal.
